(19) United States
(12) Patent Application Publication
(10) Pub. No.: US 2023/0028487 A1
(43) Pub. Date: Jan. 26, 2023

(54) THERMOACOUSTIC IMAGE-GUIDED MICROWAVE THERAPY SYSTEM

(71) Applicant: ARIZONA BOARD OF REGENTS ON BEHALF OF THE UNIVERSITY OF ARIZONA, Tucson, AZ (US)

(72) Inventors: Russell Witte, Tucson, AZ (US); Hao Xin, Tucson, AZ (US); Diego Martin, Tucson, AZ (US)

(21) Appl. No.: 17/949,866

(22) Filed: Sep. 21, 2022

Related U.S. Application Data
(62) Division of application No. 16/098,624, filed on Nov. 2, 2018, now abandoned.
(60) Provisional application No. 62/330,659, filed on May 2, 2016.

(51) Int. Cl. A61N 1/40; A61N 5/02; A61B 18/00; A61B 90/00; A61B 17/00

(52) U.S. Cl. CPC A61B 18/1815; A61N 5/025; A61N 1/403; A61B 2018/00809; A61B 2018/1853; A61B 2090/378; A61B 2018/1884; A61B 2018/00642; A61B 2017/00106; A61B 2018/00577; A61B 2017/008; A61B 2018/00529; A61B 2018/00446; A61N 2005/027; A61B 2017/00053; A61B 2018/00333

(57) ABSTRACT

A method and system capable of applying microwave therapy guided by thermoacoustic imaging and/or thermoacoustic thermometry is disclosed. The system includes a thermoacoustic imaging system and/or a thermoacoustic thermometry system that generate(s) a map of a region of interest; and a microwave therapy system that targets the region of interest using the map, and that applies the microwave therapy to the targeted region of interest. Treatment of the targeted region of interest may be employed by the microwave therapy system using real-time feedback from the thermoacoustic imaging system and/or the thermoacoustic thermometry system. Imaging and therapy may be automatically co-registered.

### CROSS REFERENCE TO RELATED APPLICATION(S)

This application claims priority to U.S. provisional patent application No. 62/330,659, filed on May 2, 2016, which is hereby incorporated herein by reference in its entirety.

### GOVERNMENT SPONSORSHIP

None.

### FIELD OF THE INVENTION

Embodiments are in the field of microwave therapy. More particularly, embodiments disclosed herein relate to systems and methods for applying microwave therapy guided by thermoacoustic imaging and/or thermoacoustic thermometry.

### BACKGROUND OF THE INVENTION

Advantages and Limitations of Breast Conservation Therapy (BCT)

Improved breast cancer screening protocols and technologies over the last two decades have led to earlier detection of breast cancer. Studies following breast cancer patients for more than 20 years report that BCT performed equally well as radical mastectomy. Although lumpectomy followed by radiation therapy is the BCT standard of care for small tumors, such treatment is still considered aggressive, leading to severe breast asymmetry in 35% of patients and morbidity in another 14%, primarily due to bleeding or infection. Surgery can also lead to life-threatening complications, particularly in patients over 70 years with comorbidities. Locally aggressive higher-stage cancers also benefit from BCT following neoadjuvant chemotherapy and tumor down-staging. These patients benefit from a superior cosmetic appearance and better prognosis when combined with radiation therapy. For later-stage cancers, including those involving metastasis, there is also emerging evidence that surgery and radical mastectomy diminish activity of natural cancer-killing cells and cytotoxicity of lymphocytes. These studies suggest that preserving healthy tissue, while destroying the tumor, may help protect against recurrence and/or distant metastasis. For these reasons, there has been a progressive trend towards minimally invasive methods with less morbidity and better cosmetic appearance for treating breast cancer. Despite the reported benefits of BCT, current protocols still require invasive surgery, and there is still a need for less invasive strategies to treat localized breast cancer, while preserving as much healthy breast tissue as possible.

Hyperthermia for Treatment of Early-Stage Breast Cancer

Over the last two decades, several thermal techniques (extreme cold or heat) have been studied and developed, including cryoablation, radiofrequency ablation (RFA), interstitial laser therapy (ILT), and high intensity focused ultrasound (HIFU) to treat solid breast cancers with the goal of obtaining efficacy similar to or better than BCT. These techniques use thermal ablation to destroy a tumor and its margins. Compared to surgery and other aggressive treatment options, thermal therapies have less scarring, better tissue preservation, superior cosmesis, faster recovery time, and lower healthcare cost. In fact, ongoing phase II clinical trials are evaluating the efficacy of cryoablation of low risk breast cancers (Luminal A) as a replacement for breast surgery. Although cryoablation is extremely effective in causing cell death for small ductal tumors <2 cm, its main drawback is the limited control of the size of the ablation zone near the metal probe and, as a result, cryoablation has not proven effective for breast tumors >1.5 cm. ILT and RFA are minimally invasive percutaneous techniques that deliver intense heat (>55° C.) to destroy small tumors. However, the RFA electrodes do not easily penetrate hard, fibrous tissue and control of the thermal dose remains challenging. Finally, noninvasive HIFU has been used to treat early-stage breast cancer by sequentially targeting multiple regions inside the tumor with a high power focused ultrasound beam near 1 MHz. Although this approach has great promise as a completely noninvasive technique for ablating small breast tumors with feedback, magnetic resonance thermometry is required to track tissue temperature, which dramatically increases the complexity, time, and cost of the procedure. Moreover, HIFU ablation for larger breast lesions (>2 cm) is still an area of research and development. Thus, despite the promise of hyperthermia for treatment of breast cancer, specific technological challenges remain, especially control of the thermal dose at the tumor site, while minimizing damage to surrounding healthy tissue, and monitoring progress during treatment. None of these approaches have demonstrated efficacy for treating larger solid breast tumors (>2 cm).

In short, there is a great need for an integrated system that combines hyperthermia treatment for localized breast cancers with real-time imaging to accurately monitor the heat zone and track temperature. Such feedback could help optimize treatment and improve outcomes for breast cancer patients. To address this unmet and urgent need, in an embodiment, the inventors propose developing and testing a novel system capable of focused microwave therapy integrated with 3D spectroscopic thermoacoustic imaging and/or thermoacoustic thermometry with millimeter resolution capability for, inter alia, mapping breast dielectric properties, tracking temperature, and/or monitoring the ablation zone during treatment.

Exemplary Challenges

1) Revolutionize treatment regimens by replacing interventions that have life-threatening toxicities with regimens that are safe and effective; and 2) Improve prognosis and reduce chance of recurrence by preserving healthy tissue and retaining natural cancer-killing cells.

Thus, it is desirable to provide embodiments of a method and system capable of applying microwave therapy that utilize thermoacoustic imaging and/or thermoacoustic thermometry guiding techniques that do not suffer from the above drawbacks.

These and other advantages of the present invention will become more fully apparent from the detailed description of the invention hereinbelow.

### SUMMARY OF THE INVENTION

Embodiments are directed to a system capable of applying microwave therapy guided by thermoacoustic imaging and/or thermoacoustic thermometry. The system comprises: a thermoacoustic imaging system and/or a thermoacoustic thermometry system that generate(s) a map of a region of interest; and a microwave therapy system that targets the region of interest using the map, and that applies the microwave therapy to the targeted region of interest.

In an embodiment, the microwave therapy system is a focused microwave therapy system, and the applied microwave therapy is focused microwave therapy.

In an embodiment, the targeted region of interest comprises breast, liver, brain, or other type of tissue.

In an embodiment, the map is generated using one or more microwave frequencies from the thermoacoustic imaging system and/or the thermoacoustic thermometry system.

In an embodiment, the microwave therapy system is an ablation microwave therapy system capable of performing ablation, and the applied microwave therapy comprises ablation.

In an embodiment, the microwave therapy system is a microwave therapy system capable of performing hyperthermia at temperatures not sufficient to ablate tissue.

In an embodiment, the system above may further comprise an ultrasound system which is co-registered with the thermoacoustic imaging system and/or the thermoacoustic thermometry system to generate the map. The ultrasound system may be a type selected from the group consisting of pulse-echo ultrasound, ultrasound shear wave imaging (such as elastography), Doppler ultrasound, acoustoelectric imaging, and a combination thereof.

In an embodiment, the microwave therapy system is configured to use integrated feedback from the thermoacoustic imaging system and/or the thermoacoustic thermometry system, while targeting the region of interest. The integrated feedback may be in real-time.

In an embodiment, the thermoacoustic imaging system and/or a thermoacoustic thermometry system comprise(s) a transducer/probe used for generating the map, wherein the microwave therapy system is configured to target the region of interest using the transducer/probe.

Additional embodiments and additional features of embodiments for the system and method for applying microwave therapy guided by thermoacoustic imaging and/or thermoacoustic thermometry are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It's understood, however, that the inventive concepts disclosed herein are not limited to the precise arrangements and instrumentalities shown in the figures. The detailed description will refer to the following drawings in which like numerals, where present, refer to like items.

FIG. 1 is a diagram illustrating thermoacoustic imaging (TAI). Portion A shows thermoacoustic effect. Portion B shows pressure induced by absorption of a microwave pulse for spherical absorber. Portion C shows in vivo TA image of a human breast and tumor (arrows) at 434 MHz.

FIG. 2 is a diagram illustrating components of proposed image-guided FMT system (top view). Left: Array of antennas creates a focused microwave beam for localized heating in the breast (a=ablation zone [>60° C.]; b=margin [50-60° C.]; and c=healthy breast tissue [<50° C.]). The figure illustrates an integrated imaging platform for real-time feedback during FMT: Spectroscopic thermoacoustic imaging and thermometry (Middle) and ultrasound imaging of tissue structure (Right) (d=broadband (1-3 GHz) horn antenna; e=custom 2 MHz ultrasound array).

FIG. 3 is a diagram illustrating a design broadband phased array element. The left portion of the figure is a top view of an antenna on a dielectric substrate with partial ground plane. In the right portion, a simulated antenna return loss (S11) indicates a broad operating range (1 to 3.5 GHz), per Aim 1/Task Ib below.

FIG. 4 is a diagram illustrating an FMT phased array and beam focusing algorithm. From top left is illustrated: an antenna array surrounding a breast model separated by coupling liquid; individual broadband monopole antenna radiator; phased array transmitting circuit; and beam focusing using STI generated 3D dielectric map, per Aim 1/Task 1a below.

FIG. 5 is a diagram illustrating a 3D printing process of realistic human breast phantom. The figure depicts an outer shell and various molds that will be printed and filled with appropriate materials to resemble heterogeneous dielectric property of breast tissue (also see FIG. 13).

FIG. 6 is a diagram illustrating a time-domain model of bioheating. Temperature in simple breast phantom with 1 cm tumor during 3 minutes of FMT. Surrounding media is mineral oil.

FIG. 7 is a diagram illustrating a robust STI modeling package using state-of-the-art simulation software (GEMS). The figure has several portions: A) model includes realistic numerical breast phantom obtained by dynamic contrast-enhanced magnetic resonance imaging (DCE-MRI), microwave excitation apparatus (signal generator, waveguide antenna), ultrasound detectors and coupling fluid. B) Conductivity map based on DCE-MRI of a breast from a patient (XY slice). C) Corresponding SAR map of same XY plane. D) Corresponding TA image at 3 GHz. E) Simulated TA spectroscopy for highly malignant (top line) to non-malignant tissue (e.g., fat; bottom line). Blood and calcifications also have very different absorption profiles in the 1-3 GHz range. Arrow in D) denotes location of tumor.

FIG. 8 is a diagram illustrating integration of two high peak power microwave transmitters into one broadband source (1.2-3.1 GHz).

FIG. 9 is a diagram illustrating a face of custom 1.5D US array (2 MHz) capable of 3D STI and US imaging.

FIG. 10 is a diagram illustrating TA imaging separates of muscle from fat. Left Column of FIG. 10: Photograph of bovine muscle (m) and fat (f) sample; Middle Column of FIG. 10: 2D pulse echo (PE) image of sample; and Right Column of FIG. 10: Co-registered 2D thermoacoustic (TA) image at 2.9 GHz (4 kW, 500 nsec). The 1 MHz single-element US transducer was mechanically scanned along green dashed line (left column). Fat has a much lower dielectric constant than muscle and appears as a shadow in the TA image and a bright signal in the PE image due to fat's higher US scattering coefficient. The sealed waveguide (MW) filled with mineral oil helped couple microwaves to the sample. The bottom row displays a second scan after placing a thin layer of muscle on top of the sample. Note bright signal at vertical arrow due to presence of tissue with high water content (muscle).

FIG. 11 is a diagram illustrating the calculation of water/fat ratio based on frequency dependence of TA signal from 2.7-3.1 GHz (4 kW, 0.3 μsec). For a given microwave intensity (I), the change in TA pressure (p) as a function of frequency (v) is directly proportional to the change in absorption (α) according to the differential equation,

The frequency

$$\frac{\partial p ( r , 𝓋 )}{\partial 𝓋} = \frac{β {c}_{s}^{2} I ( r )}{2 {C}_{P}} \frac{\partial α ( r , 𝓋 )}{\partial 𝓋} .$$

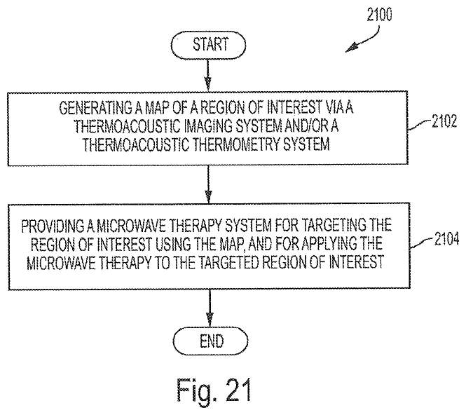

dependence of other physical parameters (thermal expansion and specific heat Cr) are negligible in soft tissue. Left portion of FIG. 11: Cross section of four tube samples containing different % water and fat. Top to bottom (left portion): Pulse echo and TA images at three microwave frequencies. Right portion of FIG. 11: Prediction of water content based on frequency dependent slope of TA signal (ΔTA/GHz) and slope of absorption coefficient (α) of water (Δα/GHz). Bar=2 mm.

FIG. 12 is a photograph of 3D printed breast phantom made of soft polymer. Occlusions have controllable dielectric properties ranging from fat to water.

FIG. 13 is a diagram illustrating temperature dependence of TA signal in water.

Amplitude decreased 1.5%/° C. during cooling. A 1 MHz ultrasound receiver was used to detect the TA signal. μwaves=2.9 GHz, 300 nsec pulses, 4 kW peak. The sample was uniformly heated to 55° C. Temperature during cooling was measured with a thermal probe.

FIG. 14 is a flowchart and procedure for STI-guided FMT platform tested in Aim 3 (below) in human cadaver breasts (post mortem).

FIG. 15 is a diagram illustrating STI simulations. Top row of FIG. 15: Conductivity map extracted from DCE-MRI (CAD file) of patients with different types of breast density (i.e., % water content or water/fat ratio). Class 1-4: <25%, 25-50%, 50-75%,>75% (glandular tissue). Bottom row of FIG. 15: Simulated STI at 2.5 GHz for Classes 1-4 in top row. The intensity provides an estimate of breast density (i.e., water content). For this example, the Class 4 breast has a density 3.9 times greater than the Class 1 breast. Spectroscopic images depend on both the water/fat ratio and breast density, a known risk factor for breast cancer.

FIG. 16 is a diagram illustrating exemplary equipment for an experiment with TA imaging/thermometry and microwave heating.

FIG. 17 is a diagram illustrating two plots of pulse echo and thermoacoustic during cooling.

FIG. 18 is a diagram illustrating two plots of thermoacoustic differential signal and thermoacoustic slope.

FIG. 19 is a diagram illustrating two plots of pulse echo and thermoacoustic during cooling.

FIG. 20 is a diagram illustrating two plots of thermoacoustic temperature map and thermoacoustic slope.

FIG. 21 is a flowchart illustrating an embodiment of a method for applying microwave therapy guided by thermoacoustic imaging and/or thermoacoustic thermometry, in accordance with an embodiment.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical thermoacoustic imaging system, thermoacoustic thermometry system, typical microwave therapy system, or typical method for applying same. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It's intended that all such additional devices, systems, methods, features, and advantages be protected by the accompanying claims.

For purposes of this disclosure, the explanations and illustrations provided throughout this disclosure for thermoacoustic imaging systems and methods for applying same may be applicable to thermoacoustic thermometry systems and methods for applying same, and may be used interchangeably.

There are potentially many different applications for the technology/system described in this disclosure, although the breast is what is being focused on. There's a need for this type of therapy. The technique is capable of providing feedback for the therapy. And this has been one of the main problems with conventional microwave therapy in that it was not possible to control the beam in a way that would not endanger healthy tissue, and not affect surrounding tissue, while being able to reach the temperature that is desired for the therapy. And so the imaging technique is related to thermoacoustic imaging which, in itself, has been known recently. But it's basically a technique that uses a microwave pulse, so very little temperature change related to the imaging technique is experienced in the imaging region. It is therefore a very safe, and non-invasive way to image. The primary contrast for thermoacoustic imaging is related to the type of microwave absorption properties of the tissue. And so, the idea is that you use a short microwave pulse that leads to, based on the thermoacoustic effect, a small amount of heating. So this is much less than a degree Celsius, and that causes thermoelastic expansion, and the expansion launches ultrasound or acoustic waves. And it's these sounds that are detected to form an image. The sound may be detectable with a conventional ultrasound receiver, or any custom ultrasound receiving device. And, an ultrasound image can be made that's related to the absorption of the microwave.

The primary source of contrast which people have used through thermoacoustics is looking at differences in the dielectric properties where the biggest contrast would be seen from fat versus muscle. Fat has a low dielectric, does not absorb microwaves very much. Muscle has a lot of water content and therefore absorbs microwaves strongly. It turns out that the signal is also very sensitive to temperature. As you're heating the tissue and it's getting warmer, especially if it's got a lot of water in it like most soft tissue, the signal is increasing as you are delivering the thermal dose, which in this case would be from a separate microwave, of course or microwave beam that's doing the heating. The idea would be to be able to monitor the heating from a microwave source that could be focused into the tissue or into the breast, but at the same time you are using this imaging modality to get feedback related to the temperature as well as any changes in the dielectric property during the heating.

Because, when you reach certain temperatures, there are changes in the dielectric properties. Although that's a secondary sort of thing that the inventors have looked at. In short, the present inventors are building a system which can focus a microwave beam down to, depending on the frequency (e.g., approximately 1-3 GHz), for example a 1-2 cm, focusing on a resolution of approximately, for example, a millimeter, which seems to be a reasonable expectation for most applications, although that could increase or decrease depending on what the application is and how deep to penetrate. For the breast, 1-2 mm would be more than enough to track temperatures on that scale and in that type of tissue. This is all occurring at the same time while being able to, in real-time, monitor the temperature and the dielectric properties of the targeted (and/or surrounding) tissue.

The thermoacoustic signal comes from the absorption of a microwave pulse that generates down, and then you detect, you only receive the sound. And then that sound is related to the absorption of the microwave. The thermoacoustic signal is also proportional to temperature. And that's the main focus. However, you have to have an ultrasound device to detect the sound, and so most ultrasound systems are detectors and also capable of sending out sound. That's your standard ultrasound imaging, pulse echo ultrasound. And that's giving you a completely different source of contrast related to the structure of the tissue. Just like when you go get an ultrasound exam. So the idea would be to have a standard ultrasound system as part of this integrated into the thermoacoustic imaging as well. So it's not like the inventors necessarily have a separate procedure here. It's all integrated together, but different modalities and different information is obtained from each of the techniques that could be used in real time with the focus microwave therapy for feedback. As an option, the ultrasound device may operate as more than just a detector. It may be used as a transmitter for different modalities or different contrasts associated therewith. Therefore, you wouldn't need to have a separate ultrasound transmitter, and you still could do all the thermoacoustic functions. However, if you transmit from the same ultrasound device that you're receiving, that would then allow you to do pulse-echo imaging. And so, the system would have all of these different techniques combined into one system. This is just one option as you don't even have to have the ultrasound.

In terms of when using pulse-echo imaging, this type of imaging has to perform two-way travel at the speed of sound, with it going down and coming back up. Whereas, the thermoacoustic imaging is only one-way travel. The microwave is speed of light, and the sound coming back is one-way travel. The present invention may just overlay them, they're automatically co-registered and so it just basically gives you free, useful information to have that available.

The thermoacoustic image could be made, which could just be a static image using, for example, one scan. And then the temperature of the sample may be changed, track the signals over time as the temperature is changing, and then obtain a temperature map. And then the present invention may also do pulse-echo on the same sample, and then may combine them if desired.

Studies and trials all over the world have been done using focused ultrasound combined with magnetic resonance (MR) scanner, and there's some conditions that are approved in the United States. For example, uterine fibroids have been approved for a long time. But they've been doing things with Parkinson's Disease, epilepsy, and different types of cancers such as bone cancer, breast cancer, and prostate cancer. With ablation therapy systems, they're capable of ablating tumors in the head by ablating parts of the brain related to Parkinson's, by using a focused ultrasound beam. This is a competing technology, and the way that they get temperature feedback, is by having it inside an MR scanner. And the MR scanner is very good at measuring temperature, via MR thermometry or thermography. However, with this system, a MR system is required which increases the complexity and cost by a significant amount.

Another problem with the ultrasound used with the MR scanner is that you're dealing with a focal spot of about a millimeter, maybe a couple of millimeters, and if you have a larger tumor, it can be very difficult to just ablate it. It can take too long, because you have to do one dose at a time, and then you have wait for things to cool down and then perform another dose. So, it can take a couple hours or more even for relatively small areas that you're ablating. There might be other disadvantages of ultrasound, for example, when going through air interfaces, or going through a bone. Bone interfaces are a problem with the ultrasound/MR therapy at least for the ultrasound. So, the applications for the ultrasound/MR therapy are indeed limited, when you need to go deeper and/or you need a larger spot.

Embodiments described in this disclosure are capable of being used on the breast but also in other areas of the body, such as the liver or kidney where you can get penetration deep into the body and be able to ablate if you utilize a feedback system such as described in this disclosure. Full body imaging and therapy may also be contemplated within the scope of this disclosure. It should be noted that the inventors are not just proposing ablation, they are also describing hyperthermia in general. So there are different types of uses for hyperthermia where you're not destroying the tissue, but you're just raising it five degrees or ten degrees. Usually within five or ten degrees. For cancer and tumors, tumor cells can't handle temperatures within the five to ten degrees for a long period of time whereas healthy cells can. So hyperthermia involving just elevated temperatures is an example of how the system may be being used to treat cancer. In embodiments below, the disclosure describes having a large tumor where you can heat it two degrees while you're delivering chemotherapy, and that will actually help shrink the tumor more because you've elevated the temperature.

The tumor then shrinks down and the surgeon can go in and remove the tumor. The way that's done now is more with radiation, instead of using hyperthermia. They would use radiation with chemotherapy to shrink the tumor down and then surgically remove the tumor. So this system described in this disclosure would not require radiation, and it might be just as effective or better. So there are different ways the technology could be used, for example, neuropathic pain for treatment of nerves, etc., where temperature is used as a form of treatment. So, you can definitely broaden this system beyond the breast.

With this technology, in any of the embodiments below, a key aspect is that the present invention is using the focused microwave beam for the heating. And so one might say, well if ultrasound is using their technology in an MR scanner, could the microwave also be used in an MR scanner for temperature mapping? This might be possible, however there are a lot of hurdles that have to be overcome, simply because you are inside the large, powerful magnet and you're trying to deliver a beam with a lot of electronic components that can interfere with (or be interfered by) the imaging system. No one has figured out how to do this inside a coil or a magnet.

Embodiments may perform thermoacoustic imaging of tissue during microwave therapy of the breast, or any other type of body tissue such as the heart, brain, or peripheral nerves.

Embodiments are directed to a system capable of applying microwave therapy guided by thermoacoustic imaging and/or thermoacoustic thermometry. The system comprises: a thermoacoustic imaging system and/or a thermoacoustic thermometry system that generate(s) a map of a region of interest; and a microwave therapy system that targets the region of interest using the map, and that applies the microwave therapy to the targeted region of interest.

In an embodiment, the microwave therapy system is a focused microwave therapy system, and the applied microwave therapy is focused microwave therapy.

In an embodiment, the targeted region of interest comprises breast, liver, brain, or other type of tissue.

In an embodiment, the map is generated using one or more microwave frequencies from the thermoacoustic imaging system and/or the thermoacoustic thermometry system.

In an embodiment, the microwave therapy system is an ablation microwave therapy system capable of performing ablation, and the applied microwave therapy comprises ablation.

In an embodiment, the microwave therapy system is a microwave therapy system capable of performing hyperthermia at temperatures not sufficient to ablate tissue.

In an embodiment, the system above may further comprise an ultrasound system which is co-registered with the thermoacoustic imaging system and/or the thermoacoustic thermometry system to generate the map. The ultrasound system may be a type selected from the group consisting of pulse-echo ultrasound, ultrasound shear wave imaging (such as elastography), Doppler ultrasound, acoustoelectric imaging, and a combination thereof.

In an embodiment, the microwave therapy system is configured to use integrated feedback from the thermoacoustic imaging system and/or the thermoacoustic thermometry system, while targeting the region of interest. The integrated feedback may be in real-time.

In an embodiment, the thermoacoustic imaging system and/or a thermoacoustic thermometry system comprise(s) a transducer/probe used for generating the map, wherein the microwave therapy system is configured to target the region of interest using the transducer/probe.

Embodiments are also directed to a method for applying microwave therapy guided by thermoacoustic imaging and/or thermoacoustic thermometry. FIG. 21 is a flowchart illustrating an embodiment of a method 2100 for applying microwave therapy guided by thermoacoustic imaging and/or thermoacoustic thermometry, in accordance with an embodiment. In an embodiment, the method comprises: generating a map of a region of interest via a thermoacoustic imaging system and/or a thermoacoustic thermometry system (block 2102); and providing a microwave therapy system for targeting the region of interest using the map, and for applying the microwave therapy to the targeted region of interest (block 2104).

In an embodiment, the microwave therapy system is a focused microwave therapy system, and the applied microwave therapy is focused microwave therapy.

In an embodiment, the targeted region of interest comprises breast, liver, brain, or other type of tissue.

In an embodiment, the step of generating the map comprises using one or more microwave frequencies from the thermoacoustic imaging system and/or the thermoacoustic thermometry system.

In an embodiment, the microwave therapy system is an ablation microwave therapy system for performing ablation, and the applied microwave therapy comprises ablation.

In an embodiment, the microwave therapy system is a microwave therapy system for performing hyperthermia at temperatures not sufficient to ablate tissue.

In an embodiment, the step of generating the map comprises using an ultrasound system which is co-registered with the thermoacoustic imaging system and/or the thermoacoustic thermometry system. The ultrasound system may be a type selected from the group consisting of pulse-echo ultrasound, ultrasound shear wave imaging (such as elastography), Doppler ultrasound, acoustoelectric imaging, and a combination thereof.

In an embodiment, the microwave therapy system uses integrated feedback from the thermoacoustic imaging system and/or the thermoacoustic thermometry system, while targeting the region of interest. The integrated feedback may be in real-time.

In an embodiment, the thermoacoustic imaging system and/or a thermoacoustic thermometry system comprise(s) a transducer used for generating the map, wherein the microwave therapy system targets the region of interest using the transducer.

### Justification for Overarching Challenges

1) Replace aggressive/life-threatening interventions with safe, effective interventions. STI-guided FMT could replace breast surgery as an effective BCT for early stage T1/T2 breast cancers and outperform other thermal therapies, including cryoablation, which has not shown benefit for tumors >1.5 cm in clinical trials. For advanced stage T3/T4 breast cancer, the imaging and therapy platform could employ mild hyperthermia (45-50° C.) to enhance neoadjuvant chemotherapy and promote tumor down-staging. This approach has improved the outcome for invasive breast cancers by reducing or eliminating possible residual tumor cells following surgery.
2) Improve prognosis and reduce chance of recurrence by preserving healthy tissue and retaining natural killer cells (e.g., cytotoxic lymphocytes). There is evidence that thermal therapies, as opposed to surgery, stimulate an antitumor immune response that could reduce the chance of recurrence and/or distant metastasis.

### Opportunity for New Technology

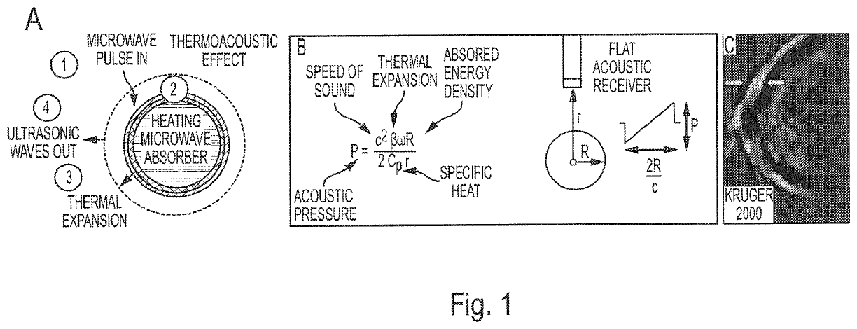

B.3. Focused Microwave Therapy (FMT) for Breast Cancer: Advantages and Limitations. Noninvasive focused microwave therapy (FMT) is a cutting-edge approach for treating breast cancer that employs an array of antennas to focus electromagnetic energy deep into tissue at GHz frequencies. This technique offers different options for treating breast cancer. For small T1/T2 stage cancers, FMT ablates the tumor and its margins as a possible replacement for breast surgery. FMT offers better penetration and larger ablation zones (>2 cm) than ILT, RFA and cryoablation. In addition, because FMT depends on tissue dielectric properties, microwaves preferentially heat and damage high-water content breast carcinomas instead of healthy breast tissue. Clinical studies have demonstrated benefits of FMT for breast conservation therapy. For larger, higher stage T3/T4 cancers, FMT delivers local hyperthermia (45-50° C.) to shrink the tumor with heat-activated neoadjuvant chemotherapy and tumor down-staging. This approach has also been suggested to facilitate local drug delivery with thermosensitive microcarriers and control of gene therapy using heat-sensitive promoters. Despite the promise of microwave therapy for treating and possibly curing solid tumor breast cancers, certain limitations have slowed its translation to the clinic and prevented widespread implementation. The first drawback is the inability to monitor local tissue temperature and provide feedback during treatment. Temperature monitoring is crucial for optimizing thermal dose at the target, while maintaining safe temperatures in surrounding healthy tissue. In addition, optimal and precise focusing of the microwave beam requires an accurate representation of the diverse dielectric properties of breast tissue. The inventors propose to address and overcome these limitations by combining broadband (1-3 GHz) FMT with thermoacoustic imaging and thermometry to help plan treatment protocols and adaptively control the thermal dose at the region of interest, especially the focal size and penetration depth, while minimizing residual heating to nearby healthy tissue.
B.4. Thermoacoustic Imagine (TAI) as Feedback for FMT: Microwave-induced thermoacoustic imaging (TAI) is an emerging modality in the biomedical field with implications for early cancer detection and treatment monitoring. It relies on the absorption of a short microwave pulse followed by transient heating, thermoelastic expansion and generation of ultrasonic waves, which are detected to form an image (FIG. 1A). The equation that relates acoustic pressure P to key physical properties for a spherical absorber is depicted in FIG. 1B. The resulting ultrasound image has contrast proportional to the absorption of the microwave pulse—a parameter that varies by several orders of magnitude depending on the microwave frequency and type of tissue. As a noninvasive hybrid modality, TAI benefits from the high resolution of ultrasound imaging (<1 mm) and high contrast of microwave imaging. Several TAI systems have been developed and tested, including a clinical scanner for diagnostic breast imaging. However, these early systems were limited by the thermoacoustic technology available at the time (single microwave frequency, long pulses, low resolution, etc.). The inventors propose several key enhancements to thermoacoustic imaging, including spectroscopy for measuring water/fat content and thermometry for tracking tissue temperature, to provide adaptive feedback during FMT for breast cancer. By estimating tissue dielectric properties and monitoring temperature, TAI could provide closed-loop feedback during FMT, optimize thermal dose at the tumor site and its margins, as well as safe exposure to surrounding healthy breast tissue.
B.4a Spectroscopic Thermoacoustic Imaging (STI) for Planning and Optimizing FMT: The inventors hypothesize that STI spanning a wide range of frequencies will better quantify breast dielectric properties compared to a single frequency system and, thereby, help plan and optimize FMT. The inventors' rationale is based on the unique absorption spectra of different types of normal and cancerous breast tissues in the GHz range. According to previous studies, the permittivity of normal and malignant breast tissue differs anywhere from 10 to 400%. It is clear that microwave absorption in the breast depends on the amount of water, adipose and other types of tissue present. To take advantage of the diverse dielectric properties of the breast for imaging, the inventors propose developing the first broadband Spectroscopic Thermoacoustic Imaging (STI) system (1.2-3.1 GHz) integrated with FMT. The STI platform would enable 3D mapping of breast dielectric properties, including breast density and water/fat ratio, at high spatial resolution (˜1mm³) as a template for planning FMT and optimizing delivery of the microwave beam to the target.
B.4b Thermoacoustic Thermometry for Guiding FMT: Monitoring temperature at and near the ablation zone is critical to ensure delivery of the proper thermal dose. Underheating may not fully destroy the tumor or kill all the cancer cells. Overheating may lead to adverse effects on healthy tissue near the tumor target. Unexpected changes in position or registration could also endanger the patient. Therefore, adaptive feedback and guidance during thermotherapy is crucial. Several techniques for noninvasive temperature monitoring have been reported, including MRI, ultrasound and infrared thermography, although each has its limitations. MRI requires a bulky magnet and expensive; ultrasound is weakly sensitive to temperature changes, while infrared thermography has poor penetration into the human body. The TA signal may also be used for real-time thermometry in tissue due to the strong dependence of the conversion of heat to acoustic pressure on water temperature (defined by the Grueneisen parameter). A recent study reported that the TA signal per ° C. increases ˜5% in water, saline and tissue. The measurements were accurate with sensitivities at 0.15° C. Indeed, thermoacoustic thermometry could provide a valuable metric for guiding FMT of breast cancer without the need of a large magnet or invasive thermocouples. This technique will be fully tested and integrated into the STI-guided FMT platform. The present invention may be considered the first broadband 3D STI system for mapping breast dielectric properties to optimize FMT and real-time 3D thermometry near the ablation zone for feedback during FMT.

### Objectives

1) Design and build broadband, image-guided FMT system that is noninvasive, safe and effective for treating small or large solid breast tumors as a possible replacement or complement to invasive surgery.
2) Benchmark performance and optimize STI-guided FMT platform using numerical breast phantom, animal tissue, and pilot study in human cadaver breasts (metrics: accuracy/precision estimating water/fat ratio, temperature, size/location/depth of microwave focus and ablation zone)

### Specific Aims

Specific Aim 1. Design, build and test platform for broadband FMT of the human breast. A 32-element phased array antenna operating from 1 to 3 GHz is designed, fabricated and tested for focused microwave treatment of breast cancer. Broadband FMT would allow greater flexibility for treating tumors. Near field focusing algorithms are initially applied to a realistic breast model with known dielectric and verified with full-wave EM modeling. EM simulations also feed a time-domain thermal model for predicting 3D temperature and dosimetry in tissue with validation using thermocouples placed in the sample. Expectations: Within 10% of model predictions, focal diameter smaller than 1 cm at 3 GHz, location accuracy within ½ focal beam diameter, temperature range exceeding 60° C. (temperature required for tissue necrosis).

Specific Aim 2. Develop and test cutting-edge thermoacoustic technology for image-guided FMT. The first broadband, high resolution spectroscopic thermoacoustic imaging (STI) is developed and tested for later integration into the FMT platform described in Aim 1. First, the inventors tested the accuracy of 3D STI for estimating the dielectric properties (water/fat ratio) in a breast phantom and animal tissue with known dielectric properties. Second, the inventors determined the accuracy and precision for tracking temperature with real-time TA thermometry. The inventors' hypothesis is that the unique features of STI would provide valuable feedback to predict energy deposition in tissue, optimize location of the beam, and ensure proper thermal dose during FMT. To accomplish these goals, the inventors integrated their existing thermoacoustic system with a custom ultrasound array capable of 3D real-time STI and pulse echo imaging near the ablation zone/region of interest. Performance Metrics: accuracy and precision estimating water/fat ratio and mapping temperature.
Specific Aim 3. Perform benchmark testing and proof-of-concept studies in human cadaver breasts. The STI and FMT systems from Aims 1 and 2 are combined for benchmark testing first in a 3D-printed numerical breast phantom and animal tissue and, later human cadaver breasts. The 3D STI maps feed the FMT beam-focusing algorithm to determine the optimal excitation (amplitude and phase) of each array element. Bench-top experiments then so determined the accuracy of STI for real-time monitoring of temperature during focused microwave heating. STI thermometry are compared with temperature measurements recorded by thermocouples embedded in the sample. Performance Metrics: accuracy for mapping temperature during FMT, size/location of the ablation zone with image guidance compared to tissue slice photography and model predictions. For the human cadaver breasts, the size of the ablation zone and water/fat ratios may then be correlated with immunohistochemistry.

### Research Strategy

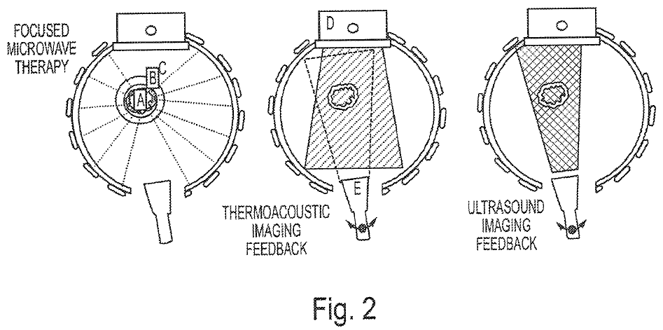

This ambitious three-year project would develop and demonstrate an integrated platform capable of closed-loop hyperthermia of the human breast. FIG. 2 illustrates the main components of the system. Aim 1 would focus on developing the FMT system; Aim 2 would develop the imaging platform (STI and ultrasound); Aim 3 would integrate FMT with imaging feedback and perform benchmark testing and proof-of-concept experiments in tissue-mimicking phantoms and human cadaver breasts. An optimized STI-guided FMT system would dramatically enhance treatment for breast cancer by avoiding side-effects of breast surgery, reducing chances of recurrence and/or metastasis, and improving cosmesis and overall quality of life for patients.

Specific Aim 1. Design, build, and test platform for broadband FMT of the human breast: This aim focuses on EM modeling, instrumentation development, and evaluation of a system capable of FMT between 1-3 GHz. While other FMT systems operate at a single frequency (e.g., 0.915 GHz), the multi-frequency system allows greater flexibility in focusing a microwave beam to different depths and focal sizes. The STI system (Aim 2) may be integrated into the FMT platform for image-guided hyperthermia and ablation therapy (Aim 3). Performance metrics: size and location of focus, temperature control at region of interest (ablation zone, margins), power delivery vs thermal dose; compare to model predictions. Define: # of elements, target temperature at ablation zone/margins, frequency [symbol]
Task 1a. Devise beamforming algorithm for FMT planning and optimization: The 3D distribution of dielectric properties (both dielectric constant and conductivity) of the sample under study is estimated from the STI image. The 3D dielectric distribution may then be used to calculate the excitation phase and amplitude of each array element to focus the microwave energy at the desired location in the sample. Two beam-focusing algorithms, namely time reversal and forward beamforming, may be studied and compared.

Time reversal, a popular method in ultrasonics, may be used to focus microwave energy at the target location (e.g., tumor) and minimize energy at other locations. For this algorithm, a virtual source is located at the position of the target, and electromagnetic wave propagation from the source to each element of the antenna array is calculated using a finite-difference time-domain (FDTD) method based on the dielectric distribution of the breast. The phase of the electromagnetic signals are recorded at each array element. The conjugate of these phases produce the required phase delays to electronically steer and focus the microwave beam back to the target. Forward beamforming is a robust algorithm for non-invasive focusing of microwave energy to the desired position in the breast. In fact, even with a rough, homogenized estimate of the breast dielectric properties, an accurate beam focal spot is achievable. This approach consists of two steps. First, a propagation model is constructed from a FDTD EM breast simulation; next, the beamformer, consisting of a finite-impulse filter (FIR) in each channel of the antenna array, is designed to maximize the microwave energy delivered at the desired spot using the propagation model constructed.

Performance Metrics: The beamforming algorithm may first be verified by full-wave numerical model, which may predict focal size, location, and intensity, including side lobes (all frequency dependent).

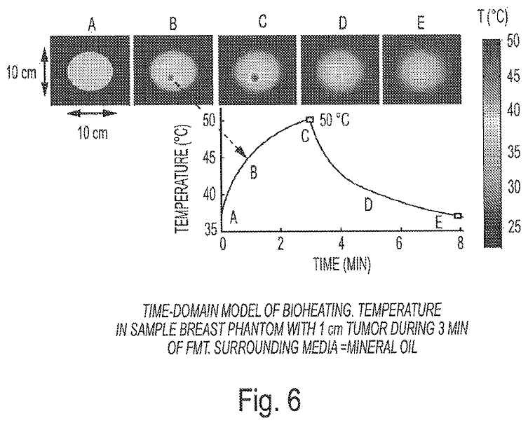

Dosimetry: Because neither beam-focusing method guarantees safe power densities away from the target, a full-wave 3D EM model may be developed, including the antenna array, to test realistic breast phantom models of different tissue density, and the surrounding environment (i.e., the matching fluid, etc.). This model better describes thermal energy deposition and temperature rise in the entire breast. A 3D FDTD thermal model similar to that reported may also be developed with the calculated thermal potential (W/m³) from the EM model, so that the temperature rise may be calculated before and during treatment with safety considerations (see FIG. 6 for FMT modeling example of bioheating of a solid breast tumor).

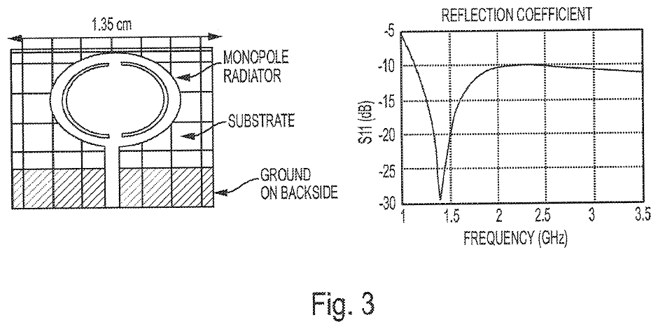

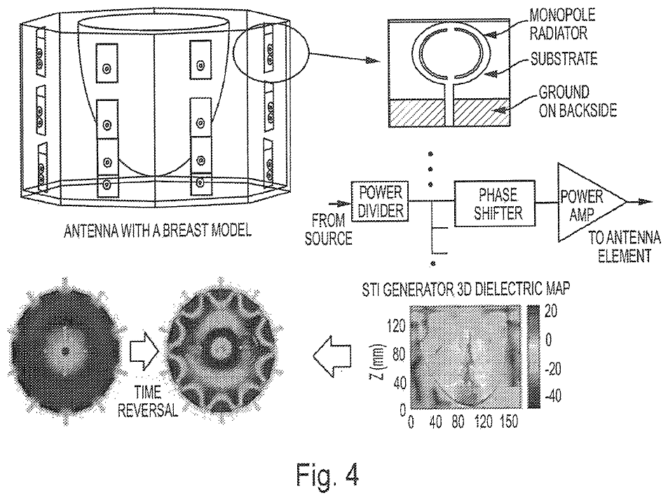

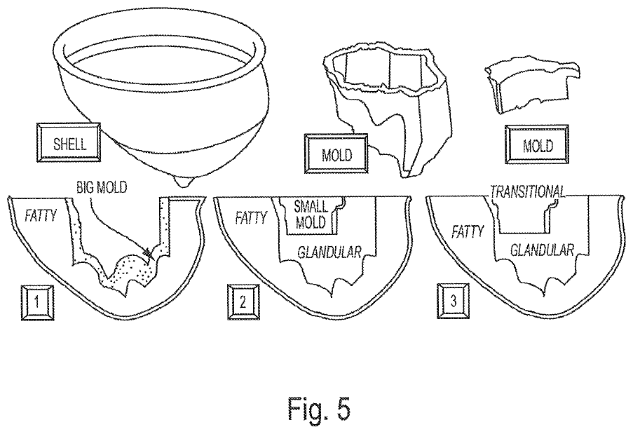

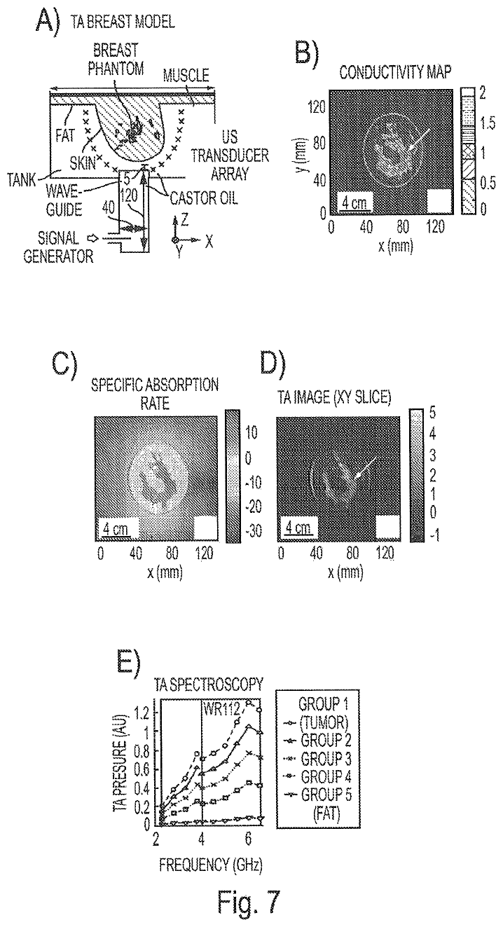

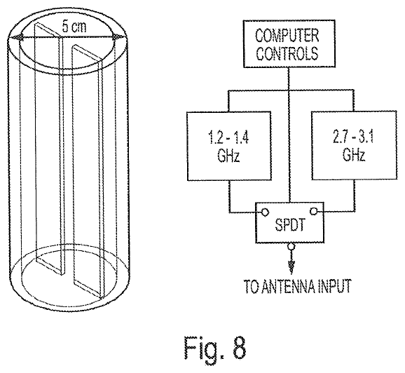

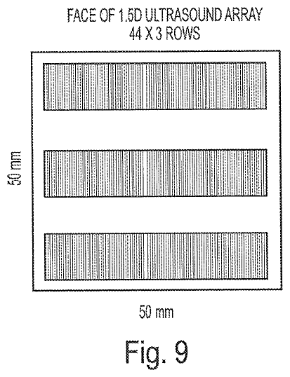

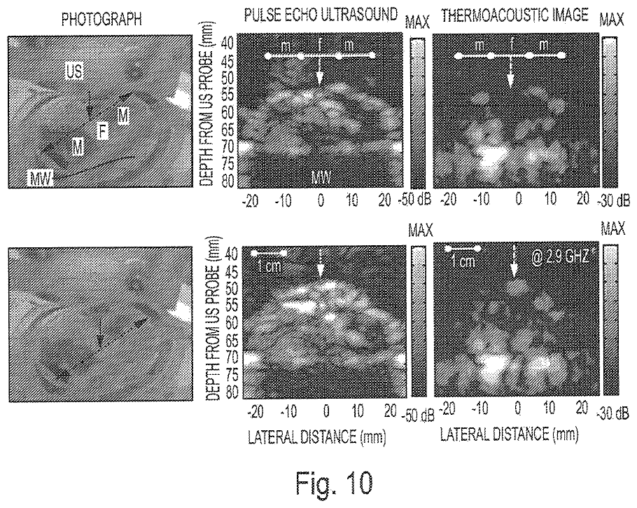

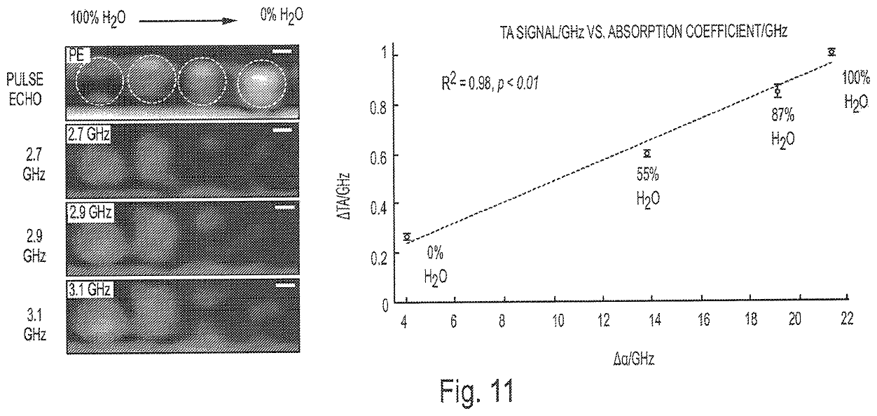

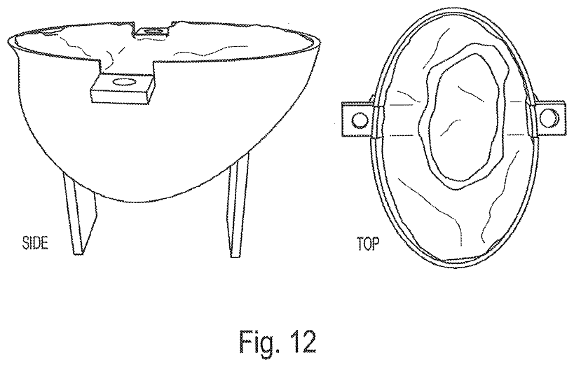

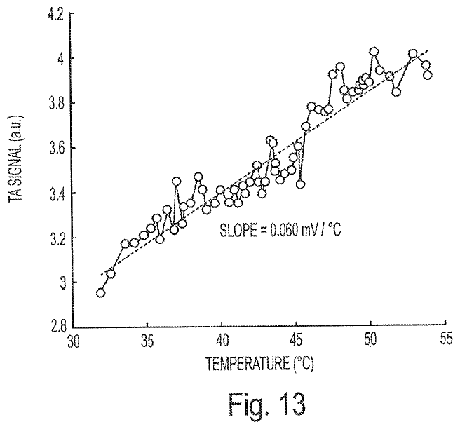

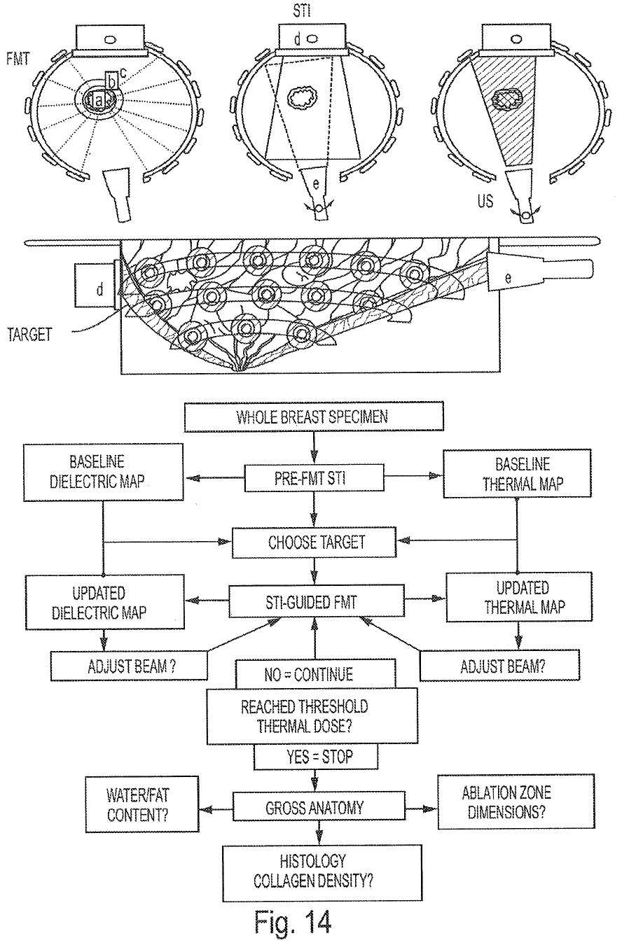

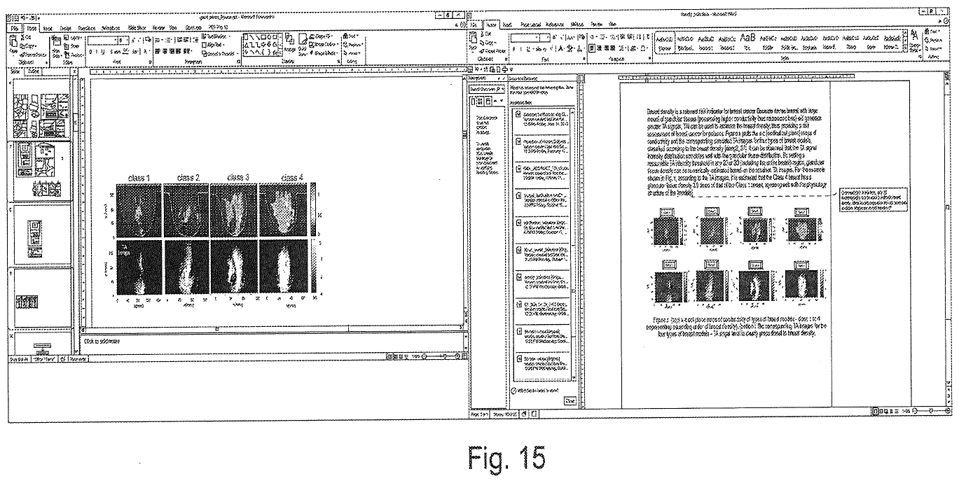

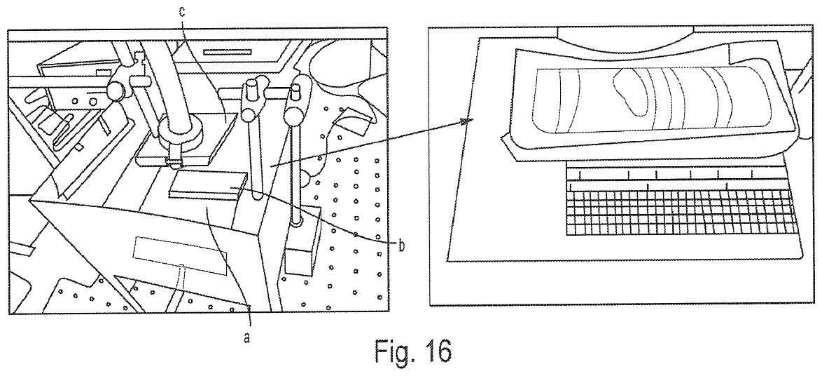

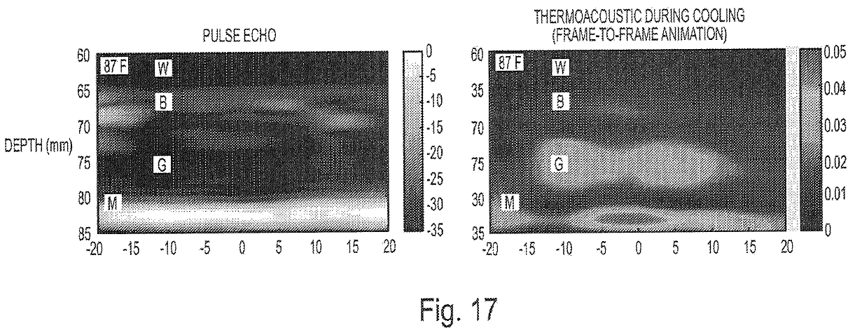

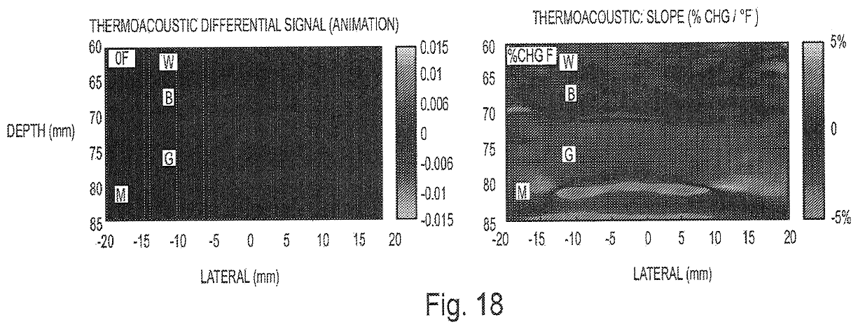

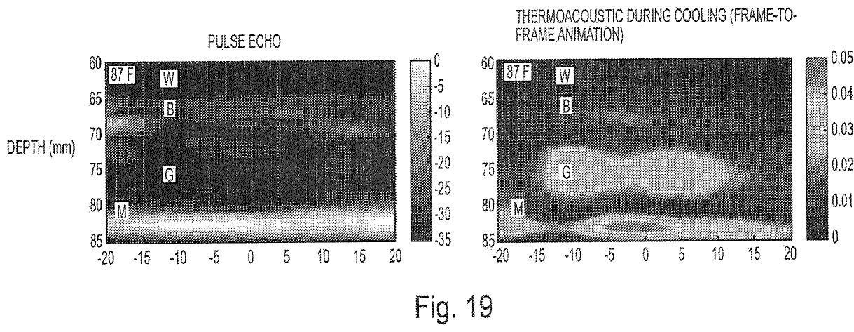

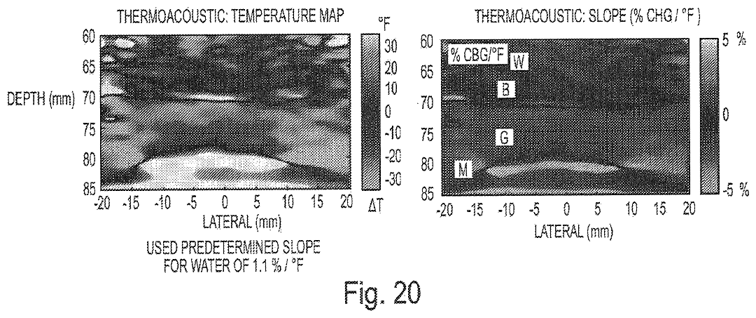

Task 1b. Design and build solid-state broadband array for microwave delivery to the breast: The inventors built a broadband platform operating between 1 and 3 GHz, which is designed to provide greater flexibility delivering microwave energy to the breast at different depths in comparison to the standard single frequency FMT system. Broadband array element: FIG. 3 shows an example of an ultra-wide-band (UWB) monopole antenna, a good candidate for the FMT array due to its simplicity and compactness. The design of the antenna array surrounding the breast model is displayed in FIG. 4. Both cylindrical and more conformal configurations (e.g., multiple circular rings with different radii) are explored to optimize the beam-focusing property. Each ring consists of an array of elements on a flexible substrate to conform to different breast sizes. Considering the size of the individual antenna elements and typical geometry of the breast, it is estimated that an array with 32 elements distributed evenly around the breast sample should be sufficient. The expected focal spot may be less than 10 mm and even smaller at 3 GHz. Based on the full-wave EM model developed, the present invention may also estimate the optimal operating frequency for different breast densities because the microwave focusing performance is affected by the microwave frequency and scattering properties of the breast. For example, according to the EM models, higher frequencies perform better in less dense breasts or for treating shallow tumors, because the smaller wavelength produces a finer focal spot and does not impact deeper tissue due to larger attenuation; conversely, tissue scattering dominates in denser breasts, suggesting lower microwave frequencies are favorable for focusing and treating deeper tumors suggesting lower microwave frequencies are favorable for focusing and treating deeper tumors.
1-3 GHz transmitting circuitry: For each antenna element, the inventors designed and built a phased array transmitting circuit using commercial-off-the-shelf (COTS) components including phase shifters, tunable attenuators and power amplifiers (FIG. 4). It is estimated that the power output of each element may be up to a few Watts. The entire array may be driven by a signal generator with two 1-to-16 power dividers (i.e., MECA 816-S-1.900-M01). All the phased array circuit components may be selected to operate within the targeted 1 to 3 GHz frequency range. The designed antenna array may be fabricated in-house using a standard printed circuit board (PCB) process. The final assembled phased array may be designed to work with a matching layer for optimal microwave delivery to the breast.
Performance Metrics: Individual array elements may be tested using a vector network analyzer to verify antenna performance (return loss and gain pattern). Based on simulations with impedance matching, the inventors expect >80% efficiency within the frequency band. Each transmitting circuit chain may be tested against the desired output phase and power ranges.
Task 2. Evaluate focusing and steering of microwave beam in numerical breast phantom: This task may verify hardware operation and assess focusing capability of the FMT system in a breast phantom and fresh animal tissue. Initial tests may involve only the coupling fluid. A microwave beam ranging from 1 to 3 GHz may be focused to different locations in the tank using the algorithms described previously. A small transducer/probe may be employed to measure the electric field distribution in the test fluid. Moreover, the measured E-field profile of the entire sample may be compared with the simulated E-field to further validate the model and hardware design. In addition to the simple liquid sample, the inventors designed and fabricated a realistic 3D-printed breast phantom with properties similar to a human breast. PI Xin's group has exploited advanced 3D printing technology for a variety of applications including breast phantom. FIG. 5 illustrates the 3D printing process for making the breast phantom. A thin outer shell (skin) and several inner molds are printed using a polymer jetting rapid prototype printer. They are then assembled with different tissue-mimicking materials representing fatty, transitional and glandular tissues, respectively (each mold is removed after the layer below cures). These materials are made from a mixture of oil, water and surfactant. The dielectric properties of each compartment may be controlled by varying the relative concentrations. The final assembled breast phantom may then be tested with FMT and compared with a time-domain heating model (FIG. 6). Although much simpler than a real human breast, the 3D breast phantom may enable initial verification of the proposed FMT hardware and algorithms.
Performance Metrics: The accuracy of the beamforming algorithm (R² and MSE) may be calculated by comparing the expected (modeled) and actual (measured) positions of the focal spots. Focus spot size at different depths as a function of microwave frequency may be evaluated and compared to model prediction. The temperature distribution in the test sample as a function of heating time may be measured using thermocouples. These measurements may be compared with simulated temperature changes (FIG. 6).
Other Pitfalls/Alternatives for Aim 1: If models or experiments indicate that the thermal dose may exceed preset limits in healthy tissue outside the target, the present invention may compensate by reducing power densities in the focal zone or increasing cooling intervals.
Specific Aim 2. Develop and test cutting-edge thermoacoustic technology for image-guided FMT: The inventors propose several significant enhancements to thermoacoustic imaging technology, including: 1) Spectroscopic capability between 1 and 3 GHz; 2) Short microwave pulses for better spatial resolution (<1 mm); 3) Integration with open platform ultrasound system and custom array for real-time 3D STI and pulse echo imaging for complementary tissue contrast (microwave absorption and structure); and 4) Thermometry; The inventors expect these hardware enhancements to more accurately quantify tissue dielectric properties (breast density, water/fat ratio), track temperature, and monitor the ablation zone during FMT. The inventors' thermoacoustic modeling package may be combined with bench-top experiments to predict and optimize the performance of the imaging system.
Task 1. Employ thermoacoustic modeling to predict outcomes and validate bench-top experiments: Accurate modeling of EM propagation is critical for estimating microwave energy deposition in tissue, ensuring safe delivery of microwaves, and predicting the thermoacoustic pressure. Thermoacoustic models typically employ acoustic wave propagation with finite element modeling and simple plane wave excitation for the microwave propagation; however, because tissue is heterogeneous and exhibits frequency-dependent absorption, this assumption may not always be accurate for estimating local microwave absorption, especially for large samples like the breast. This project may instead implement thermoacoustic imaging modeling that considers both EM and acoustic propagation through the sample. Relevant system parameters may be tested for their effect on image quality, including the feed antenna, impedance matching, coupling fluid, tissue properties, microwave frequency and pulse excitation waveform. FIG. 7 illustrates a TA model of the human breast (7A), along with simulations from a realistic breast model (3D dielectric distribution of human breast obtained by MRI) (7B-7E). The model was able to accurately reconstruct the microwave absorption profile in the breast model and predict the frequency-dependent signature (spectroscopy) of different tissues. The inventors also recently published computational studies of breast phantoms with tumor inclusions. The modeling software may be used at one or more microwave frequencies to optimize the system parameters for spectroscopic thermoacoustic imaging (STI), predict outcomes, and validate bench-top experiments.
Task 2. Build broadband source for spectroscopic thermoacoustic imaging (STI) between 1 and 3 GHz: The inventors have access to two state-of-the-art tunable microwave delivery systems capable of STI between 1.2-1.4 GHz and 2.7and 3.1 GHz. The first is a travelling wave tube amplifier (1.2-1.4 GHz, 18 kW peak, 0.05-10 μsec pulses). The second is a pulsed magnetron source (Epsco™ PG5KB, tunable: 2.7-3.1 GHz, 4 kW peak, 0.3-3 μsec pulses). These microwave transmitters may be combined into a single platform for broadband STI with unique advantages. First, the integrated system may be tunable between 1.2 and 3.1 GHz, enabling voxel-wise estimates of water/fat ratios in a heterogeneous sample. Second, the system may feature short microwave pulses down to 50 ns, yielding better spatial resolution (<1.0 mm). Third, the high pulse repetition rate (10-100 KHz) allows for fast averaging for better sensitivity (SNR). As FIG. 8 illustrates, a high power single-pole double-throw (SPDT) microwave switch (T5-523A20, Ducommun) combines the two sources. The microwave excitation system may be computer-controlled for seamless operation across the desired frequency range. Expectation: EM models predict better than 90% excitation efficiency when using an appropriate matching liquid between the waveguide and breast surface.
Task 3. Integrate broadband STI source with ultrasound system and custom ultrasound array: The inventors' existing STI platform uses single-element focused ultrasound receivers. These broadband transducers range from 0.5 to 70 MHz, corresponding to a spatial resolution from 3 mm to <0.1 mm. To overcome limitations with slow mechanical scanning of single element detectors, this project proposes real-time STI using an open platform ultrasound system (Verasonics, Inc) and custom ultrasound array. The inventors may work with FOCUS simulation software in the design and fabrication of the transducer/probe for this application. Initial designs suggest that a 1.5D array (e.g., 43 columns×3 rows) with a center frequency of 2.0 MHz and 70% fractional bandwidth may provide a resolution between 0.5 and 1 mm and able to scan a large region of interest in the breast during FMT. The probe may also be capable of 3D imaging with limited steering in the elevation direction. For a larger field of view in the breast as necessary, the ultrasound probe may be able to swivel from a fixed position (FIG. 2, right). Real-time 2D/3D imaging may be acquired at frame rates >100 Hz, limited by the repetition rate of the microwave source and safe exposure for imaging.
Alternatives: Although the 1.5D array would be designed to work only with the Verasonics system, the inventors also have a backup option using a portable clinical ultrasound scanner (zOneUltra, Zonare Medical Systems) and a standard 128-element linear array capable of 2D B mode thermoacoustic imaging at high frame rates (>100 Hz). Three linear arrays are available with different center frequencies (2, 7, and 10 MHz). The inventors have previously demonstrated photoacoustic imaging and spectroscopy in small animals and humans using a pulsed laser source and the Zonare scanner. A linear scan of the array would produce volumetric images in <5 seconds per microwave frequency. The system would also be able to acquire ultrasound images co-registered with STI (ultrasound and microwave interleaved) for complementary contrast of tissue structure.
Task 4. Quantify imaging parameters of STI in tissue-mimicking phantoms and bovine tissue. Spatial resolution, sensitivity (signal-to-noise ratio (SNR)), and depth of penetration (1/e) of the imaging platform may first be predicted with the TA modeling software (See Aim 2/Task 2) and then verified with bench-top experiments in simple phantoms (e.g., thin copper wire cross hairs embedded in Agarose gel) and fresh tissue (e.g., strips of fat buried in muscle tissue, as illustrated in FIG. 10). Expectations: based on results of their robust simulation software, initial bench-top experiments, and the initial design of the STI system, the inventors expect to achieve an axial spatial resolution (full-width half-maximum) between 0.5 and 1 mm, an SNR of at least 20 dB, and a depth of penetration between 4 and 9 cm (depending on the microwave frequencies, peak power, and pulse width, as well as bandwidth of the ultrasound array). Although penetration is primarily limited by microwave attenuation in breast tissue, most early-stage solid breast tumors are within 3 cm of the surface of the skin.

Alternatives: If either the modeling predictions or experiments indicate that the present invention may not meet expectations within the range of values listed above, then the present invention may trade off system parameters (e.g., a lower frequency ultrasound transducer/probe and longer microwave pulse width would improve sensitivity and penetration at the cost of lower spatial resolution). Even with a resolution of 2 mm, the present invention imaging platform may still have much better resolution compared to the size of the focal beam for FMT (<10 mm at 3 GHz).

Task 4a. Estimate dielectric properties with STI as a template for optimizing FMT beamforming. Because the TA signal is proportional to microwave absorption, it may be used to estimate the dielectric properties of breast tissue. It is well known that breast tumors usually have higher water content than normal, fatty breast tissue, which would help 1) quantify breast density and 2) distinguish tumors from healthy adipose tissue. Such “dielectric breast maps” would also be valuable feedback to optimize near-field beamforming during FMT (see Aim 1). FIG. 10 demonstrates that water-based tissue (e.g., muscle or tumor) produces a much stronger TA signal than fat-based tissue (e.g., adipose). In addition, TA imaging studies between 2.7 and 3.1 GHz revealed that the amplitude and spectrum of the TA signal may be combined to accurately estimate water content. The amplitude and spectrum at each pixel is correlated with the known absorption spectra of water and fat based on a linear mixture model (FIG. 11). Estimating water content with STI may be further tested for accuracy and reliability in breast phantoms and fresh tissue.

Water/fat ratios may be measured with STI between 1.2 and 3.1 GHz in both tissue mimicking phantoms with known dielectric properties and bovine tissue with a known % fat rating. The inventors' 3D-printed numerical breast model (see FIG. 12 and Aim 1) has acoustic and dielectric properties that may be controlled by varying the concentrations of oil, water and surfactant in each compartment. The water/fat ratio at each pixel is calculated from a linear model of frequency-dependent TA images (see FIG. 11) and compared with the sample's known properties as validation or to improve the imaging technique. In Aim 3, these dielectric maps may help determine the required excitation phases and amplitudes for focusing the beam during FMT.

Expectations/Alternatives: The inventors expect the measured water-fat ratios to be within one standard deviation or 20% of the true or predicted values. If the inventors do not achieve this level of success, the inventors may then consider increasing the microwave peak power or degree of averaging (spatial and temporal) until the required accuracy is achieved.
Task 4b. Demonstrate TA thermometry and develop real-time temperature monitoring algorithm. Thermoacoustic imaging could provide real time non-invasive temperature monitoring based on the strong temperature dependence of the Grueneisen parameter Γ for water, which describes the conversion from heat (due to microwave absorption) to pressure. Moreover, Γ is approximately linear and proportional to temperature within the range used for hyperthermia. The TA pressure amplitude is expressed by \hat{p}=p(T)/p(T₀)=(1−bT₀)+bT , where p(T₀) and p(T) refer to the pressure at temperature T₀ (reference temperature) and T, respectively. The slope b is determined through a calibration process and/or estimated. Practically, b is tissue dependent (e.g., water vs. fat), so that a priori determination of the water-fat ratio obtained from the baseline STI dielectric map is included in the model. For each sample, the temperature rise distribution is calculated from the measured p(T₀) and p(T) before and after the treatment period. Temperature sensitivity of the TA signal is calculated based on the uncertainty in the mean value of the measurements (standard error), which is directly related to the SNR of the TA signal. One recent study of TA thermometry reported a sensitivity of 0.15° C. with 20 averages. The present invention preliminary results (FIG. 13) confirm these findings with the TA signal in water increasing 1.5%/° C. between 30 and 55° C.

The phantoms and tissue preparations described in the previous task may be used to measure the sensitivity and accuracy of TA thermometry in samples with non-uniform dielectric properties. The sample may be heated to 70° C. and 3D STI temperature maps may be captured as the sample cools. These measurements may be compared to thermocouple measurements recorded simultaneously. Expectations/Alternatives: Based on preliminary data, the inventors expect TA thermometry to have excellent sensitivity (<1° C.) and spatial resolution (<1 mm). If the inventors achieve this level of performance for stationary samples with an accuracy of 2° C. up to 70° C., it would be as effective or better than MR thermometry (MRT), the gold standard for noninvasive temperature mapping. If this level of accuracy is not demonstrated, the inventors may safely average over more trials (temporal averaging) or over a larger region (spatial averaging). Because ultrasound imaging and TAI also have the capability to track motion in real-time, future applications of the technology could incorporate motion tracking. A fiber optic thermometer may be considered, if the microwave beam interferes with the thermocouples (or vice versa). Finally, if the thermocouples do not provide sufficient spatial sampling, ultrasound thermometry may be implemented (using the same ultrasound transducer), which depends on changes of the speed of sound with temperature (−0.1%/° C.), for cross-validation; however, the inventors expect this technique to be less accurate than STI for large differential temperatures.

### Specific Aim 3. Perform Benchmark Testing and Proof-of-Concept Studies in Human Cadaver Breasts

As a prelude to clinical STI-guided FMT, the present invention may obtain paired unpreserved whole human female breast specimens from fresh autopsies for post-mortem imaging, ablation therapy, and analysis. The breast itself may be intact except for the outermost skin layer, which may remain to cover the body surface. Whole breast specimens may allow us to further develop and validate STI-guided FMT as an effective technique for breast conservation therapy. FIG. 14 illustrates the STI-guided platform with the flow chart and procedure used in Aim 3.

Task 1a. The FMT and STI systems developed and tested in the previous aims may be combined into a single platform for benchmark testing and proof-of-concept studies in human cadaver breasts. A multichannel trigger device (Quantum Composer may control the timing of image acquisition, thermometry, and microwave heating.
Task 1b. Develop an iterative STI-guided FMT algorithm and procedure. In order to ablate a small T1/T2 tumor or shrink a large T3/T4 tumor and limit thermal damage to surrounding healthy tissue, it is necessary to incorporate a tuning mechanism that would adaptively focus the microwave beam spot at the desired position. With the aid of STI-based thermometry, adaptive focusing and steering may be achieved by:
1) Before FMT, acquire 3D STI and ultrasound images to identify the position of target (tumor) and plan therapy. The STI image also provides a reference for thermometry.
2) Employing the focusing technique described in Aim 1/Task 1, adjust the amplitude and phase of each antenna element in the phased array so that the calculated beam spot is focused at the target [e.g., the tumor position (ρ₀, φ₀, z₀)].
3) Start FMT at low power. After a short interval of mild heating, repeat an STI scan of the sample to obtain the latest temperature change distribution. The position where the strongest signal appears should correspond with the actual beam focal spot. Assume the new location is at (ρ₁, φ₁, z₁).
4) Adjust the phase and amplitude of the phased array elements so the theoretical beam spot now focuses to (ρ₀-Δρ, φ₀-Δφ, z₀-Δz), where Δρ=ρ₁−ρ₀, Δφ=φ₁−φ₀ and Δz=z₁−z₀.
5) Repeat steps 3 and 4 until the beam spot is at the desired position.

First, the 3D breast phantom and tissue preparations described in Aim 2/Task 4 may be used to test the iterative STI-guided FMT procedure and determine the accuracy and speed of the feedback system. Once efficacy is demonstrated in these samples, the adaptive algorithm may be applied to the human cadaver breast. Temperatures (up to at least 70° C.) may be simultaneously recorded using thermocouples.

Expectations/Alternatives: Thermocouples placed near the target may be problematic. First, the wires may alter the E-field and produce hot spots in the breast. Second, the thermocouple only provides point measurements inside a large volume. The present invention may address these issues by modeling the effect of the thermocouple at different locations in the sample to verify that no sites outside the target exceed a preset threshold maximum temperature. Also, between FMT intervals (i.e., during timeout periods for imaging and cooling), the thermocouples may be repositioned close to the target for thermal feedback and validation with the bioheat model (see FIG. 10). Use of fiber optic temperature sensors is another option. Finally, if the breast phantom proves too difficult for controlling dielectric properties (e.g., the inventors observe >25% MSE between experiments and simulations), the present invention may include employing fresh bovine tissue with different % fat ratings.
Task 2a. Identify location of target and generate baseline dielectric map in human cadaver breasts. Whole breast specimens (prescreened for incidental lesions see Aim 3/Task 3) may be weighed, vacuum-sealed in a thin layer of plastic to simulate skin, suspended in Agarose™ gel, and placed in imaging chamber (FIG. 14, top). An initial volumetric STI scan may estimate the dielectric properties of each breast specimen prior to FMT and identify lesions or potential targets in the breast for FMT. Ultrasound images co-registered with STI may also be acquired as an anatomical reference. Simulations for different breast types are shown in FIG. 15. Results from STI may be compared with gross anatomical and histologic analysis for validation and to assess accuracy and sensitivity of STI for estimating water/fat ratios. STI may also be compared with histochemical analysis of formalin-preserved paraffin or frozen tissue sections stained for collagen (fibrous tissue) with Masson's trichrome stain and for fat with Oil Red O (Sudan Red). Image analysis of the photographic breast tissue slices may provide an independent estimate of fat and collagen content near the ablation zone. Expectations: The inventors expect their beamforming algorithm to tolerate inaccuracies of 25% for estimating water/fat ratios. Since the resolution of STI is much smaller than the focal spot of FM, spatial averaging may be used to improve SNR.
Task 2b. Test STI-guided FMT procedure on human cadaver breasts: Initial dielectric maps from the previous task may be used as a template for focusing and steering the microwave beam. The inventors would normally expect only ˜15% (or 3 out of 20) of the breast specimens to contain incidental pathologic lesions. To increase the yield to 10 specimens and enhance the statistical power, the breast imaging team may first prescreen cadaver breasts with a portable ultrasound scanner to identify incidental lesions that could be used as a target for FMT. For the remaining breasts that do not have incidental lesions, a coordinate (distance from skin) may be chosen as the target. The initial STI scan may provide a baseline for STI thermometry. Temperature probes inserted in the breast may provide thermal feedback near the target. Once the target coordinates have been identified, the beamforming algorithm may be combined with the iterative procedure described in Aim 3/Task 1 to optimize the location of the focal spot. STI may be interleaved between high power FMT heating pulses (i.e., during <2 minute cooling phase). These STI periods may provide updated dielectric and thermal maps that adaptively adjust the position of the focus. Once the threshold temperature (or thermal dose) at the target is achieved, FMT will stop. At least three FMT-induced lesions may be performed at different locations in each breast at depths of 1, 3 or 5 centimeters. Following FMT, the breast may be sent to pathology for gross anatomical photographic analysis of the ablation zone and surrounding tissue. The dimensions of the ablation sites may be compared to that predicted by the dielectric models and STI-derived thermal maps during FMT. Histochemical examination of formalin-preserved paraffin or frozen tissue sections stained for collagen (fibrous tissue) with Masson's trichrome stain and for fat with Oil Red O (Sudan Red) may further characterize the ablation zones. Finally, the accuracy of STI-guided FMT may be estimated as a function of tissue depth by comparing results with thermometry, simulations, and post-FMT tissue analysis.
Other Pitfalls/Alternatives for Aim 3: Blood and calcium deposits could confound the dielectric map, affect beamforming and alter heating. However, the inventors expect their effect on beam focusing to be modest because water and fat dominate breast tissue. The inventors may also add blood and calcium to their models, since their dielectric properties are known. In fact, the inventors recently calculated the properties of breast calcium (oxalate and hydroxyapatite) with TAI near 3 GHz. For future in vivo studies, blood vessels may be included in the bioheat model as cooling sources. Finally, if the breast sags or moves during STI-guided FMT, we'll consider placing the breast in a compatible gel mold for stabilization. If the STI-guided FMT platform meets expectations, the system would be modified for safety testing in humans. If the platform does not meet expectations, the inventors may consider further evaluation and optimization in large animal in vivo studies.
Future Vision: If the inventors demonstrate adaptive, precise, and accurate focusing of a microwave beam in the breast with real-time sub-mm feedback, STI-guided FMT could emerge as a promising option for minimally invasive breast cancer therapy. Noninvasive STI-guided FMT has the potential to revolutionize hyperthermia treatment for breast cancer. For early-stage T1/T2 tumors, FMT could replace breast surgery by accurately delivering intense heat to destroy the tumor and its margins while preserving healthy breast tissue with sub-cm precision. Alternatively, FMT could deliver regional hyperthermia combined with neoadjuvant chemotherapy to shrink locally aggressive T3/T4 tumors prior to resection. These approaches would not only improve the prognosis and reduce side effects of traditional aggressive treatments but could also lower the chances of recurrence, metastasis or death due to breast cancer.

### Summary of Aims and Tasks of an Exemplary Embodiment

Aims 1-4 are depicted in Table 1 below.

Aim 1. Design, build, and test platform for broadband FMT of the human breast

Task 1. Devise beamforming algorithm for FMT planning and optimization

Task 2. Design and build solid-state broadband array for microwave delivery to the breast

Task 3. Evaluate focusing and steering of microwave beam

Define: microwave frequency, input power, # elements

Determine location and size of focus, temperature at focus and margins; compared to model predictions.

Accuracy within 10% of model predictions and thermocouple measurements

Aim 2. Develop and test STI platform for image-guided FMT:

Task 1. Build broadband source for spectroscopic thermoacoustic imaging (STI) between 1 and 3 GHz

Task 2. Integrate broadband STI source into ultrasound system with custom 1.5D array

Task 3. Measure imaging performance of STI system in tissue-mimicking phantoms and fresh tissue

Calculate spatial resolution, contrast, and depth of penetration of imaging system. Calculate water/fat ratios.

Resolution within 10% of that expected by diffraction theory (˜1 mm at 1.5 MHz), penetration limited by microwave field intensity and absorption and depends on frequency. Accuracy within 10% of average water/fat content of tissue.

Aim 3. Integrate STI with FMT: test in breast-mimicking phantom and bovine tissue

Task 1. Develop real-time temperature monitoring algorithm and procedure based on STI

Task 2. Develop an iterative STI-guided FMT algorithm and procedure

Aim 4. STI-guided FMT: proof-of-concept experiments on human cadaver breasts

Task 1. Identify location of lesion/target and generate baseline dielectric map in human cadaver breasts with ultrasound imaging and STI

Task 2. Test STI-guided FMT hyperthermia procedure on human cadaver breasts

### Aim 1: Develop Hardware/Software for FMT (Antenna Array, Power Divider, Switch, Beamforming, Modeling)

Determine accuracy (±25% of beam width) for focusing and steering of microwave beam; set criteria

Aim 2: Develop Hardware/Software for STI (Broadband source, switch, ultrasound array, modeling)

Measure imaging parameters (resolution, depth of penetration) and compare with modeling predictions

Determine accuracy for estimating water-fat ratios in breast phantom and tissue with known values; set threshold criteria (±20%)

Aim 3: Combine STI and FMT hardware and software

Determine accuracy of STI Thermometry (10% of deltaT, std)

Determine accuracy of iterative algorithm for steering beam to desired location (with 5 mm of target)

Aim 4: Repeat A3/T2 on human cadaver breasts; proof of concept Location/extent of ablation (accuracy in space)

Water-Fat Ratio (accuracy)

STI Thermometry (accuracy)

TABLE 1

| | Goal | Metrics | Comparators | Threshold/Criteria |
|---|---|---|---|---|
| Aim 1 | Develop FMT | Location/size μwave focus | M, TC | ±50% BW |
| Aim 2 | Develop STI | Resolution/penetration/w-f | M, known val | <25% MSE |
| | | ratio | | |
| Aim 3 | Integrate and test | STI thermometry/iterative | TC, known | <25% MSE; |
| | STI/FMT | algor. | val | Margin < BW |
| Aim 4 | Demonstrate in | Thermometry, w-f ratio, | TC, TSP, H | <25% MSE; |
| | Human Breast | extent | | Margin < BW |

In Table 1, TC = Thermocouples, M = Model, TSP = Tissue Slice Photography, H = Histology, BW = Beam Width at Focus, MSE = Mean Square Error

### An Embodiment of an Experiment with TA Imaging/Thermometry and Microwave Heating

### An exemplary setup for TA Imaging and Thermometry is Illustrated in FIGS. 16-20

FIG. 16 is a diagram illustrating exemplary equipment for an experiment with TA imaging/thermometry and microwave heating. In FIG. 16,
a) =Matching Layer (2.7 GHz delivered from waveguide below)
b) =Sample (saline gel with bacon on top)
c) =0.5 MHz Ultrasound Transducer
   Sample was heated in microwave oven (2.4 GHz) to 87 degrees F. (measured with thermocouples)
   TA imaging and thermometry (2D B mode) performed during cooling phase down to 69 degrees F.

FIG. 17 is a diagram illustrating two plots of pulse echo and thermoacoustic during cooling. In FIG. 17,

w=water

g=gel

b=bacon

m=matching layer

FIG. 18 is a diagram illustrating two plots of thermoacoustic differential signal and thermoacoustic slope. In FIG. 18,

gₘ=1.25±0.22% Chg/° F.

bₘ=0.42±0.34% Chg/° F.

mₘ=−4.2±3.0% Chg/° F.

It is noted that the slope depends on placement of reference. In this experiment, some areas were heating and other areas were cooling at the same time (indicative of sign change of slope).

It would be best to calibrate (i.e., determine the slope) for all known microwave absorbers and then use this to estimate change of temperature based on the change of the TA signal.

FIG. 19 is a diagram illustrating two plots of pulse echo and thermoacoustic during cooling. In FIG. 19,

w=water

g=gel

b=bacon

m=matching layer

FIG. 20 is a diagram illustrating two plots of thermoacoustic temperature map and thermoacoustic slope. In FIG. 20,

gₘ=1.25±0.22% Chg/° F.

bₘ=0.42±0.34% Chg/° F.

mₘ=4.2 ±3.0% Chg/° F.

It is noted that the slope depends on placement of reference. In this experiment, some areas were heating and other areas were cooling at the same time (indicative of sign change of slope).

It would be best to calibrate (i.e., determine the slope) for all known microwave absorbers and then use this to estimate change of temperature based on the change of the TA signal.

Although embodiments are described above with reference to a thermoacoustic imaging system and/or method for applying same for generating a map of a region of interest, a thermoacoustic thermometry system and/or method for applying same may alternatively or additionally be employed. Such alternatives are considered to be within the spirit and scope of the present invention, and may therefore utilize the advantages of the configurations and embodiments described above.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1-11. (canceled)

12. A method for applying microwave therapy guided by thermoacoustic imaging, the method comprising:
generating a map of a region of interest via a thermoacoustic imaging system; and
providing a microwave therapy system for targeting the region of interest using the map, and for applying the microwave therapy to the targeted region of interest, wherein the microwave therapy system is a microwave therapy system for performing hyperthermia at temperatures not sufficient to ablate tissue, and the applied microwave therapy comprises the hyperthermia.

13. The method of claim 12, wherein the microwave therapy system is a focused microwave therapy system, and the applied microwave therapy is focused microwave therapy.

14. (canceled)

15. The method of claim 12, wherein the step of generating the map comprises using one or more microwave frequencies from the thermoacoustic imaging system.

16-17. (canceled)

18. The method of claim 12, wherein the step of generating the map comprises using an ultrasound system which is co-registered with the thermoacoustic imaging system.

19. The method of claim 18, wherein the ultrasound system is a type selected from the group consisting of pulse-echo ultrasound, ultrasound shear wave imaging, Doppler ultrasound, acoustoelectric imaging, and a combination thereof.

20. The method of claim 12, wherein the microwave therapy system uses integrated feedback from the thermoacoustic imaging system, while targeting the region of interest.

21. The method of claim 20, wherein the integrated feedback is in real-time.

22. The method of claim 12, wherein the thermoacoustic imaging system comprises a probe that emits frequencies which are applied to the region of interest and which are used for generating the map, and wherein the microwave therapy system targets the region of interest using the probe which also emits the applied microwave therapy to the region of interest.

23. A method for applying microwave therapy guided by thermoacoustic thermometry, the method comprising:
generating a map of a region of interest via a thermoacoustic thermometry system; and
providing a microwave therapy system for targeting the region of interest using the map, and for applying the microwave therapy to the targeted region of interest, wherein the microwave therapy system is a microwave therapy system for performing hyperthermia at temperatures not sufficient to ablate tissue, and the applied microwave therapy comprises the hyperthermia.

24. The method of claim 23, wherein the step of generating the map comprises using an ultrasound system which is co-registered with the thermoacoustic thermometry system.

25. The method of claim 23, wherein the thermoacoustic thermometry system comprises a probe that emits frequencies which are applied to the region of interest and which are used for generating the map, and wherein the microwave therapy system targets the region of interest using the probe which also emits the applied microwave therapy to the region of interest.
